(19) United States
(12) Patent Application Publication
(10) Pub. No.: US 2005/0064497 A1
(43) Pub. Date: Mar. 24, 2005

(54) Method for assessing the risk of cardiovascular disease

(76) Inventor: Jukka Salonen, Jannevirta (FI)

Correspondence Address: BIRCH STEWART KOLASCH & BIRCH, PO BOX 747, FALLS CHURCH, VA, 22040-0747, US

(21) Appl. No.: 10/951,902

(22) Filed: Sep. 29, 2004

Related U.S. Application Data
(62) Division of application No. 10/014,590, filed on Dec. 14, 2001, now abandoned.

(51) Int. Cl. C12Q 1/68; C12Q 1/48

(52) U.S. Cl. 435/6; 435/15

(57) ABSTRACT

The present invention is directed to a method identifying a condition in an individual in which elevation of serum or plasma HDL concentration or HDL cholesterol concentration provides enhanced protection against cardiovascular disease, the method comprising the step of testing the individual for a disorder that detrimentally affects the protective effect of HDL, whereby absence of such a disorder is an indication of enhanced protection against cardiovascular disease when said individual exhibits elevated serum or plasma HDL or HDL cholesterol concentration.

This application is a Divisional of co-pending application Ser. No. 10/014,590, filed on Dec. 14, 2001, and for which the entire contents of all are hereby incorporated by reference.

### FIELD OF THE INVENTION

The present invention is generally directed to a method for assessing the risk of cardiovascular disease (CVD), such as coronary heart disease (CHD), including myocardial infarction, and cerebrovascular disease in an individual, such as a human. Specifically the invention is directed to a method of identifying a condition in an individual in which condition an elevated serum or plasma high-density lipoprotein (HDL) concentration or HDL cholesterol concentration provides enhanced protection against cardiovascular disease. In addition, the invention provides a method of predictably treating an individual in order to enhance the plasma or serum HDL or HDL cholesterol of the said individual. Furthermore the invention provides a kit for carrying out the methods.

### BACKGROUND OF THE INVENTION

A large number of prospective population studies have shown that elevation of high-density lipoproteins (HDL) is associated with a reduced incidence of coronary events, coronary mortality and atherosclerotic progression.^{1,2} The etiologic role of HDL in atherosclerosis and CHD has not, however, been confirmed in randomized clinical trials. The reasons why HDL elevating therapies do not consistently reduce cardiovascular risk are unknown.

There is a paradigm according to which any elevation of HDL is beneficial to health.

This is, however, challenged by three lines of observations, which have been left unexplained. First, in populations with heavy alcohol intake, a high plasma HDL cholesterol concentration does not associate with reduced coronary and total mortality.³ Second, in alcoholics, a high HDL is not associated with effective reverse cholesterol transport.⁴ Third, recent reports suggest that a combination of a fibrate and cerivastatin, a HMG-CoA reductase inhibitor (Astatin@) might induce deaths, even though this combination raises HDL levels. Common to both observations is that HDL elevation is caused by general liver induction or liver damage. Statins tend to elevate hepatic transaminases in plasma.⁵ Also alcohol elevates both HDL and liver transaminase levels. A wide variety of chemicals can produce liver enlargement, peroxisome proliferation, and induction of peroxisomal and microsomal fatty acid-oxidizing enzyme activities.

An undamaged liver has phase I and phase II detoxification systems. The phase I consists of the cytochrome P450 enzymes (CYP). Mutations in the genes that encode these enzymes reduce the efficacy of the CYP system and lead to the predisposition to liver damage. Also, gene mutations in the phase II detoxification enzymes lead to an enhanced sensitivity to liver damage. The phase II enzymes are defined here to include liver enzymes such as the catalase, paraoxonases, superoxide dismutases, glutathione peroxidases, glutahione synthases, glutathione reductases, glutathione transferases, glutamyl-cysteinyl synthase, quinone reductases, diaphorases, thioredoxins, glutaredoxins, peroxiredoxins, epoxide hydrolases, aldehyde hydrolases, aldo-keto reductases, properdins, selenoproteins P and W, N-acetyl-transferases, metallothioneins, sulfurtransferases, alcohol dehydrogenases, aldehyde dehydrogenases, glutamate dehydrogenases, dihydrodiol dehydrogenases, or carboxyl esterases. DNA mutations in any of the genes encoding these proteins can cause liver damage and impair the protective function of HDL.

Variation in the response to HDL elevating drugs may be due to genetic variations that may provide a molecular basis for differences in drug metabolizing enzymes such as CYP1, CYP2, and CYP3 subtypes.

Oxidative stress and free radicals have been implicated in the etiology of a number of diseases, including cancers, coronary heart diseases and type II diabetes. The human body has a number of endogenous free radicals scavenging systems, which have genetic variability. The serum paraoxonase (PON) is an enzyme carried in the HDL that contributes to the detoxification of organophosphorus compounds but also of carcinogenic products of lipid peroxidation.⁶⁻¹⁴ PON1 is polymorphic in human populations and different individuals also express widely different levels of this enzyme.^{9,11-13}

### SUMMARY OF THE INVENTION

The object of the present invention is a method of identifying a condition in an individual in which an elevated serum or plasma HDL concentration or HDL cholesterol concentration provides enhanced protection against cardiovascular disease, the method comprising the step of testing the individual for a disorder that detrimentally affects the HDL function, i.e. the protective effect of HDL, whereby absence of such a disorder is an indication of enhanced protection against cardiovascular disease when said individual exhibits elevated serum or plasma HDL or HDL cholesterol concentration.

Furthermore, the invention is directed to a method of treatment of an individual to protect the individual against the risk of cardiovascular disease, the method comprising the steps of testing the said individual for a disorder which detrimentally affects the protective effect of HDL, identifying and selecting an individual free of said disorder, and treating the selected individual in order to enhance the HDL or HDL cholesterol level of said individual.

According to a further aspect the present invention provides a method for assessing the risk of cardiovascular disease in an individual, the method comprising the step of determining the serum or plasma HDL or HDL cholesterol concentration in said individual, and testing the individual for a disorder that detrimentally affects the HDL function, whereby identification of such a disorder is an indication of reduced protection against, i.e. an increased risk of cardiovascular disease when said individual exhibits elevated serum or plasma HDL or HDL cholesterol concentration.

In addition the invention is directed to a kit for use in the above methods, comprising means for testing the individual for a condition or disorder which affects the protective effect of HDL.

As is understood by the person skilled in the art, the HDL level in an individual can be assessed by determining the HDL concentration or a fraction thereof, e.g. the HDL cholesterol concentration of said individual.

### DETAILED DESCRIPTION OF THE INVENTION

According to the invention, a condition or disorder which affects the protective effect or function of HDL, is, for example, liver damage or a condition involving oxidative stress. Both liver damage and oxidative stress have a detrimental effect on the protective effect or function of HDL against cardiovascular disease, such as coronary heart disease, including myocardic infarction, and cerebrovascular disease.

The protective action of HDL depends on the ability of the liver to maintain the antioxidative capacity of HDL and the efficacy of HDL in the reverse transport of cholesterol from the arteries to the liver. An elevation of γ-glutamyltransferase (GGT) indicates that these functions of HDL are compromised.

A condition of liver damage in an individual can be established in many ways, a convenient method involving determination of the serum or plasma activity or concentration of an enzyme marker comprising γ-glutamyltransferase. In a condition involving liver damage, the concentration of γ-glutamyltransferase is elevated over the normal or reference values. This reference value or range can vary to some degree according to the specific methods used for determining the marker, but typically the reference value will be in the range of 20 to 100 units/L. For many purposes, a suitable value is 60 units/L. γ-glutamyltranspeptidase (EC 2.3.2.2 ) acts as a glutathionase and catalyzes the transfer of the glutamyl moiety of glutathione to a variety of amino acids and dipeptide acceptors. This enzyme is located on the outer surface of the cell membrane. It is widely distributed in mammalian tissues involved in absorption and secretion. In humans, hepatic GGT activity is elevated in some liver diseases. GGT is released into the bloodstream after liver damage.

Patients with cholestasis usually have increased serum γ-glutamyltransferase concentrations, and the concentrations may be increased by certain enzyme-inducing drugs or alcohol abuse. Measurements of serum γ-glutamyltransferase aid in interpreting elevated serum alkaline phosphatase values. γ-glutamyltransferase activity in serum is the sum of the activities of heterogeneous isoenzymes that migrate in zone electrophoresis as follows: GGT1 to the prealbumin-albumin region, GGT2 to the alpha-1-globulin region, GGT3 to the alpha-2-globulin region, and GGT4 to the beta-globulin region.

Instead of, or in addition to, measuring the γ-glutamyltransferase activity or concentration, it is possible to use genotyping of genomic DNA from a sample of said individual, and to identify mutations or polymorphisms in the DNA which influence liver damage or plasma or serum γ-glutamyltransferase activity or concentration. There are several γ-glutamyltransferase genes located on chromosome 22 and at least two of these appear to be transcribed. A third alternative is to measure the expression at the RNA level of the γ-glutamyltransferase.

The sample from which DNA can be extracted can be for example a blood sample. Genotyping can be carried out by using per se known techniques, for example PCR techniques involving the use of suitable primers and amplification systems. The geno-typing method can be amplified restriction fragment length polymorphism (ARFLP) that utilizes PCR and restriction enzyme cleavage-site recognition. Additional methods such as DNA amplification by PCR followed by minisequencing and or sequence-specific oligonucleotide probe (SSOP) analysis can also be used. Also, genotyping can be performed by using DNA microarrays or DNA chips that provide information in the same assay of a number of DNA polymorphisms that affect the liver function. It is foreseen that a large number of DNA polymorphisms such as single nucleotide polymorphisms (SNP) are determined by the use of a single DNA chip. Also the expression of the genes encoding the γ-glutamyltransferase and the phase I and II detoxification enzymes can be assayed by microarray.

Oxidative stress is another condition which has a detrimental effect on the protective effect of HDL. A suitable marker for oxidative stress is the paraoxonase enzyme. The activity or concentration of paraoxonase can be determined in a serum sample from the individual, using per se known techniques, for example based on the capacity of paraoxonase to hydrolyse paraoxon, and by monitoring p-nitrophenol formation, for example using absorbance techniques. A reduced paraoxonase activity is an indication of oxidative stress, including increased lipid peroxidation. Consequently a low paraoxonase activity is an indication that the protective effect of HDL is impaired in the individual. A reference value within a reference range of 40 to 200 nmol/ml/min is usually applicable, a typical normal value for paraoxonase activity being appr. 100 nmol/ml/min.

Instead of, or in addition to, measuring the paraoxonase activity or concentration, it is possible to apply genotyping of DNA from a sample of said individual, and identification of mutations or polymorphisms which influence plasma or serum paraoxonase activity or concentration. Two polymorphisms are currently known in human PON1. The Q191R polymorphism was the first mutation of PON1 reported.^{9,12} The second one is the missense mutation of A to T in codon 54, producing a substitution of methionine (M) to leucine (L) (Met54Leu⁸; known also as Met55Leu⁹). Both these polymorphisms have been shown to affect serum PON activity,^{12,15} and in particular, the L54 allele has been associated with an increased PON activity. A further alternative is to measure the expression of the genes encoding the PON enzyme.

According to the U.S. Pat. No. 6,242,186, homozygosity of the L54 allele in the PON1 gene protects against certain diseases associated with oxidative stress. The L allele has consistently been associated with an increased paraoxonase activity in human serum.⁷⁻¹² It was observed that there was less lipid peroxidation among men who carried the PON1 54 L allele. In such individuals, an enhanced HDL or HDL cholesterol concentration would therefore have a protective effect against cardiovascular disease. In the opposite, individuals who do not carry this mutation would not benefit from the protective effect of HDL against cardiovascular disease.

For genotyping purposes, DNA can be extracted for example from a blood sample. Genotyping can be carried out by using per se known techniques, for example PCR techniques involving the use of suitable primers and amplification systems. Such a system is described for example in the U.S. Pat. No. 6,242,186.

The antioxidative capacity of HDL can be assessed by isolating HDL from plasma or serum e.g. by ultracentrifugation or precipitation and exposing the isolated HDL to oxidizing conditions e.g. by adding to the reaction oxidative agents such as oxygen free radicals such as peroxyl radical, superoxide radical, hydroxyl radical or hydroperoxyl radical. The radicals can be generated chemically utilizing the Fenton-Haberman-Weiss reaction for instance by adding reduced transition metal such as copper or iron, by using a radical generating substance such as ABAP (2,2′-azobis(amidinopropane) dihydrochloride) or AMVN (2,2′-azobis(2,4)-dimethylvaleronitrile) or by ionizing or other radiation, UV light, heating or by other means. The resistance of the target HDL (HDL isolated from an individual being examined) can be determined as the time lag to oxidation of HDL when exposed to said radicals. The oxidation of HDL can be determined by monitoring the formation of conjugated dienes at 234 nm absorbance by a spectrophotometer or by measuring periodically the concentration of an indicator compound of oxidation. Such a compound can be an oxidized phospholipid such as lysophospatidylcholine (lysolesitine), an oxidized fatty acid such as hydroxy or epoxy fatty acid, or a cholesterol oxidation product such as hydroxy cholesterol or epoxy cholesterol or ketocholesterol. The start of oxidation of HDL or the maximum rate of oxidation can be determined. The reference values are different for different methods. As an example, if oxidation of HDL is monitored spectrophotometrically following the formation of conjugated dienes at 234 nm, and copper ions are used to induce oxidation at a concentration of 10-100 micromoles per liter, a lag time of less than 30-200 min is an indication of reduced antioxidative capacity of HDL.

Lipid peroxidation in vivo can be assessed by measuring either immunologic response to immunogenic epitopes of oxidized lipoproteins, such as antibodies to oxidized low density lipoprotein.¹⁶ Lipid peroxidation in vivo can also be assessed by measuring oxidation products of lipids or lipoproteins such as oxidized phospholipids, oxidized fatty acids, or cholesterol oxidation products.¹⁶ Oxidized fatty acids such as hydroxy and epoxy fatty acids can be measured by gas chromatography mass spectrometry or immunolochemical methods. Oxidation products of arachidonic acid such as isoprostanes can be used as indicators of lipid peroxidation in vivo. Lipid peroxidation can also me assessed by determining the proportion of electronegative LDL of total LDL by chromatographic or electrophoretic methods. Further, lipid peroxidation can be assessed by measuring plasma or serum concentration of conjugated dienes, an oxidation product of dienes. The reference values depend on the method used. As an example, plasma F₂-isoprostane levels of 20-60 ng/L or more, total plasma hydroxy fatty acids of 1-5 μmol/L or more and plasma electronegative LDL of 3-10% or more of total LDL indicate increased lipid peroxidation in vivo.

The present invention also makes it possible to treat an individual in order to protect said individual against the risk of cardiovascular disease, by identifying whether said individual is responsive to the beneficial effects of a high HDL concentration. Such a method comprises a step of determining whether said individual has a condition which detrimentally affects the effect of high HDL. If said individual is free of such a condition, such individual can be treated in order to enhance his HDL level.

Such a treatment can be a drug treatment. A suitable drug can be a drug selected from the group consisting of niacin, a statin, an apolipoprotein AI or AII synthesis enhancing agent, a PPAR alpha agonist such as fibrate, a PPAR gamma or delta agonist, a sterol absorption inhibiting agent such as a resin, a CETP inhibitor, an ACAT inhibitor, a PLTP agonist, a LCAT agonist, a lipoprotein lipase (LPL) agonist, a hepatic lipase agonist, a scavenger receptor B1 (SRB1) agonist, or an ATP-binding cassette A1 (ABC1) agonist. A statin can be for example selected from the group consisting of atorvastatin, fluvastatin, lovastatin, pravastatin and simvastatin, a fibrate can be selected from the group consisting of bezafibrate, ciprofibrate, clofibrate, fenofibrate and gemfibrozil, and a resin can be selected from the group consisting of colestipol and cholestyramin. It is, however, also possible to enhance HDL through physical activity or physical exercise.

The invention also provides for kits suitable for carrying out the methods according to the invention. Such a kit carries the necessary means for identifying a condition which affects the protective effect of HDL, such as for example the means necessary to determine enzyme, for example γ-glutamyltransferase or paraoxonase activity in a sample, such as a serum sample from the individual, or means for performing necessary genotyping of a DNA sample from said individual. In addition the kit can contain means for measuring HDL or HDL cholesterol in a sample, such as a serum or plasma sample from the said individual. Such kits preferably contain the various components needed for carrying out the method packaged in separate containers and/or vials and including instructions for carrying out the method. Thus, for example, some or all of the various reagents and other ingredients needed for carrying out the determination, such as buffers, primers, enzymes, control samples or standards etc can be packaged separately but provided for use in the same box. Instructions for carrying out the method can be included inside the box, as a separate insert, or as a label on the box and/or on the separate vials.

Experimental

In the following tests, the protective effect of HDL elevation in patients with liver damage was studied.

For assessing the protective effect, a prospective cohort study, the “Kuopio Ischaemic Heart Disease Risk Factor Study” (KIHD).^{1,2} was used. The study protocol for KIHD was approved by the Research Ethics Committee of the University of Kuopio, Finland. The study sample comprised men from Eastern Finland aged 42, 48, 54 or 60 years. A total of 2682 men were examined during 1984-89. All participants gave a written informed consent. Relevant baseline measurements were available for 2464 men. The average follow-up time was 11.4 years resulting to over 28,000 person-years of follow-up. γ-glutamyltransferase activity was determined according to the Nordic recommendation.¹⁷ The measurement of cholesterol concentration in serum lipoproteins and other risk factors, and the classification of acute coronary events and deaths have been described.^{1,2}

Among men whose liver enzyme (γ-glutamyltransferase) was within the normal range (60 IU/L or less), elevation of HDL was associated with decreased risk of acute coronary event (Table). On the average, the risk was reduced by 44% (95% confidence interval 14-68%) per each mmol/L of serum HDL cholesterol. However, in men whose liver enzyme was elevated, the risk increased 3.3-fold (95% CI 1.2 to 9.3-fold) per each mmol/L of HDL cholesterol. These relative risks differed significantly of each other (p<0.01). The addition of any measured risk factor as a covariate singly or jointly did not affect this difference. Similarly, the relative risks for coronary, all cardiovascular and all-cause death were significantly different between men who had no liver enzyme elevation and those who did (Table). There was a similar trend for cerebrovascular strokes.

As an indicator of lipid peroxidation, serum ferritin concentration was used. The study population was divided into those with normal serum ferritin (200 micrograms per liter or less) and those with elevated serum ferritin (>200 μg/L). A high serum HDL concentration was associated with a reduced cardiovascular mortality only in the subjects whose serum ferritin was normal (relative risk 0.60, 95% CI 0.33 to 1.09, p=0.095), whereas a high HDL tended to be associated with an increased risk (relative risk 1.02, 95% CI 0.37 to 2.77, p=0.976) among those with elevated serum ferritin. There were similar trends for the incidence of acute coronary events, cerebrovascular strokes and coronary deaths.

When the study cohort was stratified according to the PON1 codon 192 genotype, a high serum HDL cholesterol concentration was associated with a reduced risk of myocardial infarction only among the wild type (arginine) homozygotes (relative risk 0.04, 96% CI 0.01 to 0.19, p<0.001), whereas the associations of HDL cholesterol with myocardial infarction risk in subjects with the other genotypes were weak. There were similar trends in cerebrovascular strokes and cardiovascular and coronary deaths.

Our population-based data indicate that high serum HDL levels lose their protection against CHD among men who have liver damage, enhanced lipid peroxidation, a genotype that predisposes to liver damage or to enhanced lipid peroxidation. This effect modification was observed also for cardiovascular and total mortality, although high HDL was not protective of cerebrovascular strokes and cardiovascular and total mortality in our study. Our observations imply that an elevation of HDL is not always beneficial for human health. The liver damage and enhanced lipid peroxidation may be caused by heavy alcohol intake, drugs and hepatotoxic nutrients or contaminants in food.

Relative risk of acute coronary events, coronary, cardiovascular and any death, per 1
mmol/L of serum HDL cholesterol, in men without and with liver damage at baseline.

| | No liver damage: | | | Liver damage: | | | |
|---|---|---|---|---|---|---|---|
| | γ-glutamyltransferase | | | γ-glutamyltransferase | | | |
| | 60 IU/L or less | | | >60 IU/L | | | |
| | (n = 2253) | | | (n = 211) | | | |
| | | 95% | | | 95% | | |
| Outcome (number of | Relative | confidence | p- | Relative | confidence | p- | p for |
| men with each event) | risk | interval | value | risk | interval | value | difference |
| Acute coronary event | 0.56 | 0.32, 0.86 | 0.008 | 3.32 | 1.19, 9.29 | 0.022 | <0.01 |
| (n = 381) | | | | | | | |
| Coronary death | 0.59 | 0.28, 1.24 | 0.161 | 5.16 | 1.23, 21.64 | 0.025 | <0.01 |
| (n = 141) | | | | | | | |
| Cardiovascular death | 0.91 | 0.49, 1.69 | 0.763 | 6.01 | 1.74, 20.80 | 0.005 | <0.01 |
| (n = 187) | | | | | | | |
| All-cause death | 0.95 | 0.61, 1.48 | 0.818 | 2.46 | 1.16, 5.22 | 0.019 | <0.05 |
| (n = 370) | | | | | | | |

Cox' proportional hazards' models are adjusted for age, cigarette-years, serum apolipoprotein B (mg/L), use of antihypertensive drugs, maximal oxygen uptake (mL/kg × min), history of any atherosclerosis-related disease, family history of CHD and five examination years.

References
1. Salonen J T, Salonen R, Seppanen K, Rauramaa R, Tuomilehto J. High density lipoprotein, HDL₂ and HDL₃ subfractions and the risk of acute myocardial infarction: a prospective population study in Eastern Finnish men. Circulation 1991; 84: 129-39.
2. Salonen J T, Ylä-Herttuala S, Yamamoto R, Butler S, Korpela H, Salonen R, Nyyssönen K, Palinski W, Witztum J L. Autoantibody against oxidised LDL and progression of carotid atherosclerosis. Lancet 1992; 339: 883-7.
3. Perova N V, Oganov R G, Williams D H, Irving S H, Abernathy J R, Deev A D, Shestov D B, Zhukovsky G S, Davis C E, Tyroler H A. Association of high-density-lipoprotein cholesterol with mortality and other risk factors for major chronic noncommunicable diseases in samples of US and Russian men. Ann Epidemiol 1995; 5: 179-85.
4. Liinamaa M J, Hannuksela M L, Kesäniemi Y A, Savolainen M J. Altered transfer of cholesteryl esters and phospholipids in plasma from alcohol abusers. Arterioscler Thromb Vasc Biol 1997; 17: 2940-7.
5. Farmer J A, Torre-Amione G. Comparative tolerability of the HMG-CoA reductase inhibitors. Drug Saf 2000; 23: 197-213.
6. Mackness M I, Thompson H M, Hardy A R, Walker C H. Distinction between ‘A’-esterases and arylesterases. Implications for esterase classification. Biochem J 1987; 245: 293-62
7. Mackness M I, Arrol S, Durrington P N. Paraoxonase prevents accumulation of lipoperoxides in low-density lipoprotein. FEBS Lett 1991; 286: 152-4.
8. La Du B N, Adkins S, Kuo C L, Lipsig D. Studies on human serum paraoxonase/arylesterase. Chem Biol Interact 1993; 87: 25-34
9. Humbert R, Adler D A, Disteche C M, Hassett C, Omiecinski C J, Furlong C E. The molecular basis of the human serum paraoxonase activity polymorphism. Nature Genet 1993; 3: 73-6.
10. Davies H G, Richter R J, Keifer M, Broomfield C A, Sowalla J, Furlong C E. The effect of the human serum paraoxonase polymorphism is reversed with diazoxon, soman and sarin. Nature Genet 1996; 14: 334.
11. Mackness M I, Mackness B, Durrington P N, Connelly P W, Hegele R A. Paraoxonase: biochemistry, genetics and relationship to plasma lipoproteins. Curr Opin Lipidol 1996; 7: 69-76.
12. Mackness M I, Arrol S, Mackness B, Durrington P N. Alloenzymes of paraoxonase and effectiveness of high-density lipoproteins in protecting low-density lipoprotein against lipid peroxidation. Lancet 1997; 349: 851-2.
13. Mackness B, Durrington P N, Mackness M I. Polymorphisms of paraoxonase genes and low-density lipoprotein peroxidation. Lancet 1999; 353: 468-9.
14. Shih D M, Gu L, Xia Y- R, et al. Mice lacking serum paraoxonase are susceptible to organophosphate toxicity and atherosclerosis. Nature 1998; 394: 284-7.
15. Garin M C, James R W, Dussoix P, et al. Paraoxonase polymorphism Met-Leu54 is associated with modified serum concentrations of the enzyme. A possible link between the paraoxonase gene and increased risk of cardiovascular disease in diabetes. J Clin Invest 1997; 99: 62-6.
16. Salonen J T. Markers of oxidative damage and antioxidant protection: assessment of LDL oxidation. Free Rad Res 2000; 33 suppl.: S41-46.
17. The Committee on Enzymes of the Scandinavian Society for Clinical Chemistry and Clinical Physiology. Recommended method for the determination of gamma-glutamyltransferase in blood. Scand J Clin Lab Invest 1976; 36: 119-125.

1. A method of identifying a condition in an individual in which an elevated serum or plasma HDL concentration, or HDL cholesterol concentration, provides enhanced protection against cardiovascular disease, the method comprising the step of testing the individual for a disorder that detrimentally affects the protective effect of HDL, whereby absence of such a disorder is an indication of enhanced protection against cardiovascular disease when said individual exhibits elevated serum or plasma HDL or HDL cholesterol concentration.

2. The method according to claim 1, wherein the disorder is selected from liver damage and oxidative stress.

3. The method according to claim 1, comprising the additional step of determining the serum or plasma HDL or HDL cholesterol concentration.

4. The method according to claim 2 wherein the disorder comprises liver damage and testing for liver damage comprises determining γ-glutamyltransferase or a liver transaminase activity or concentration in a serum or plasma sample and comparing it to a selected reference value for γ-glutamyltransferase.

5. The method according to claim 2, wherein the disorder comprises liver damage and testing for liver damage comprises genotyping mutations or polymorphisms inducing or predisposing to liver damage or influencing serum or plasma γ-glutamyltransferase activity or concentration or the testing of the expression of the genes encoding these proteins.

6. The method according to claim 2, wherein the disorder comprises oxidative stress and testing for oxidative stress comprises determining serum or plasma activity or concentration of one or several phase I or phase II detoxification enzyme.

7. The method according to claim 2, wherein the disorder comprises oxidative stress and testing of oxidative stress is the assessment of serum or plasma concentration of ferritin or an oxidized fatty acid, oxidized phospholipid or cholesterol oxidation product.

8. The method according to claim 6, wherein the detoxification enzyme is a cytochrome P450 enzyme or the catalase, a paraoxonase, a superoxide dismutase, a glutathione peroxidase, a glutahione synthase, a glutathione reductase, a glutathione transferase, a glutamyl-cysteinyl synthase, a quinone reductase, a diaphorase, a thioredoxin, a glutaredoxin, a peroxiredoxin, an epoxide hydrolase, an aldehyde hydrolase, an aldo-keto reductase, a properdin, the selenoproteins P or W, an N-acetyl-transferase, a metallothionein, a sulfurtransferase, an alcohol dehydrogenase, an aldehyde dehydrogenase, a glutamate dehydrogenase, a dihydrodiol dehydrogenase, or a carboxyl esterase.

9. The method according to claim 2, wherein the disorder comprises oxidative stress and testing for oxidative stress comprises determining the antioxidative capacity of HDL

10. The method according to claim 4, wherein the reference value is selected from a reference range of 20 to 100 units per liter.

11. The method according to claim 1, wherein the cardiovascular disease is coronary heart disease or cerebrovascular disease.

12. The method according to claim 11, wherein the coronary heart disease is myocardial infarction.

13. (cancelled)

14. (cancelled)

15. (cancelled)

16. (cancelled)

17. (cancelled)

18. (cancelled)

19. (cancelled)

20. (cancelled)

21. (cancelled)

22. (cancelled)

23. (cancelled)

24. (cancelled)

25. A kit for identifying a condition in an individual in which condition an elevated serum or plasma HDL or HDL cholesterol concentration provides enhanced protection against cardiovascular disease, or for predicting an individual's response to HDL or HDL cholesterol elevating treatments, wherein the kit comprises means for testing the individual for a disorder which detrimentally affects the protective effect of HDL.

26. The kit according to claim 25, wherein the additional condition is liver damage and the means comprise means for determining serum or plasma γ-glutamyltransferase or for genotyping genomic mutations and/or polymorphisms.

27. The kit according to claim 25, wherein the additional condition is oxidative stress and the means comprise means for determining paraoxonase activity or concentration, the antioxidative capacity of HDL, or genotyping genomic mutations and polymorphisms.

28. The kit according to claim 25 for assessing an individual's risk of cardiovascular disease further comprising means for determining HDL or HDL cholesterol concentration in a serum or plasma sample of said individual.
